(19) United States
(12) Patent Application Publication
(10) Pub. No.: US 2014/0336193 A1
(43) Pub. Date: Nov. 13, 2014

(54) POTENT NON-UREA INHIBITORS OF SOLUBLE EPOXIDE HYDROLASE

(71) Applicant: The Trustees of Columbia University in the City of New York, New York, NY (US)

(72) Inventors: Donald W. Landry, New York, NY (US); Shi-Xian Deng, White Plains, NY (US); Stevan Pecic, Astoria, NY (US); Kirsten Alison Rinderspacher, New York, NY (US)

(21) Appl. No.: 14/339,183

(22) Filed: Jul. 23, 2014

Related U.S. Application Data
(63) Continuation of application No. PCT/US13/23008, filed on Jan. 24, 2013.
(60) Provisional application No. 61/590,701, filed on Jan. 25, 2012.
(60) Provisional application No. 61/590,792, filed on Jan. 25, 2012.
(60) Provisional application No. 61/650,950, filed on May 23, 2012.

(51) Int. Cl. C07D 211/96; C07D 405/12; C07D 401/12

(52) U.S. Cl. CPC C07D 211/96; C07D 401/12; C07D 405/12
USPC 514/237.2; 546/245; 435/184; 435/375; 514/330; 546/189; 514/316; 546/171; 514/314; 546/283.7; 514/321; 544/130; 546/143; 514/310

(57) ABSTRACT

The present invention relates to compounds that exhibit vasodilatory and anti-inflammatory effects by inhibiting the activity of soluble epoxide hydrolase (sEH). The present invention is also directed to methods of identifying such compounds, and use of such compounds for the treatment of diseases related to dysfunction of vasodilation, inflammation, and/or endothelial cells. In particular non-limiting embodiments, components of the invention may be used to treat hypertension.

### CROSS-REFERENCE TO RELATED APPLICATIONS

This application is a continuation of International Application No. PCT/US13/023,008, filed Jan. 24, 2013, which claims the benefit of and priority to U.S. Provisional Application Ser. No. 61/590,701, filed Jan. 25, 2012; U.S. Provisional Application Ser. No. 61/590,792, filed Jan. 25, 2012; and U.S. Provisional Application Ser. No. 61/650,950, filed May 23, 2012; each of which is hereby incorporated by reference inn its entirety, and to each of which priority is claimed.

### STATEMENT REGARDING FEDERALLY SPONSORED RESEARCH OR DEVELOPMENT

This invention was made with government support under grant No. HG003914, awarded by the National Institutes of Health. The Government has certain rights in the invention.

### 1. INTRODUCTION

The present invention relates to compounds that exhibit vasodilatory and anti-inflammatory effects by inhibiting the activity of the enzyme soluble epoxide hydrolase (sEH). The present invention is also directed to the use of such compounds for the treatment of diseases related to dysfunction of vasodilation, inflammation, and/or endothelial cell function. In particular non-limiting embodiments, compounds of the invention may be used to treat hypertension.

### 2. Background of the Invention

Epoxide hydrolases are a group of enzymes that are ubiquitous in nature, detected in species ranging from plants to mammals. These enzymes are functionally related in that they catalyze the addition of water to an epoxide, resulting in a diol. One subtype of epoxide hydrolase is the soluble epoxide hydrolase (sEH). sEH plays an important role in the metabolism of lipid epoxides. Endogenous substrates of sEH include epoxyeicosatrienoic acids (EETs), which are effective regulators of blood pressure and inflammation.

The metabolism of arachidonic acid by cytochrome P450 monoxygenase leads to the formation of various biologically active eicosanoids, and is the primary route of EET synthesis. Three types of oxidative reactions are known to occur to the precursor eicosanoids, and one of these, olefin epoxidation (catalyzed by epoxygenases), produces EETs. Four important EET regioisomers are [5,6]-EET, [8,9]-EET, [11,12]-EET, and [14,15]-EET. These arachidonic acid derivatives function as lipid mediators in certain tissues, potentially through receptor-ligand interactions, and further, can be incorporated into tissue phospholipids (Bernstrom et al. 1992, J. Biol. Chem. 267:3686-3690).

Hypertension has been shown to result from an impairment of endothelium dependent vasodilation (Lind, et al., Blood Pressure, 9: 4-15 (2000)). In healthy individuals, endothelium derived hyperpolarizing factor, EDHF, hyperpolarizes vascular smooth muscle tissue resulting in endothelium-dependent relaxation. EETs are known to provoke signaling pathways which lead to cell membrane hyperpolarization, and therefore have been considered as a candidate EDHF. In vascular tissue, hyperpolarization by EETs results in increased coronary blood flow and improved recovery of myocardium from ischemia-reperfusion injury. (Wu et al., 272 J. Biol. Chem 12551 (1997); Oltman et al., 83 Circ. Res. 932 (1998)). Accordingly, EETs are predicted to be useful in the treatment of hypertension as well as ischemia-related damage and disease.

In addition to promoting vasodilation, EETs have also been shown to exhibit anti-inflammatory properties. For example, 11,12-EET can reduce inflammation by decreasing the expression of cytokine induced endothelial cell adhesion molecules (such as VCAM-1) (Node, et al., Science, 285: 1276-1279 (1999); Campbell, TIPS, 21: 125-127 (2000); Zeldin and Liao, TIPS, 21: 127-128 (2000)). Other studies have demonstrated that EETs can inhibit vascular inflammation by inhibiting NF-κB and IκB, which prevents leukocyte adhesion to vascular cell walls. As such, EETs are also predicted to be useful in reducing inflammation and alleviating endothelial cell dysfunction (Kessler, et al., Circulation, 99: 1878-1884 (1999).

Hydrolysis of EETs by sEH converts the EETs to corresponding diols. Such diols have been shown to exhibit diminished vasodilatory and anti-inflammatory effects (Smith et al., 2005, Proc. Natl. Acad. Sci. USA. 102:2186-91; and Schmelzer et al., 2005, Proc. Natl. Acad. Sci. USA. 102:9772-7). As inhibition of sEH leads to accumulation of active EETs, such inhibition provides a novel approach to the treatment of hypertension and vascular inflammation (Chiamvimonvat et al., 2007, J. Cardiovasc. Pharmacol. 50:225-37). To date, the most successful sEH inhibitors reported are 1,3-disubstituted ureas. These urea-based inhibitors have been shown to treat hypertension and inflammatory diseases through inhibition of EET hydrolysis in several animal models. However, these inhibitors often suffer from poor solubility and bioavailability, which makes them less therapeutically efficient (Wolf et al., 2006, J. Med. Chem. 335:71-80). Therefore there remains a need for identifying new sEH inhibitors for therapeutic application.

### 3. SUMMARY OF THE INVENTION

The present invention relates to compounds of Formula I:

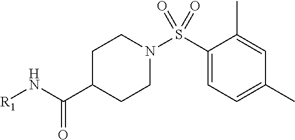

wherein R₁ is described herein below. The present invention also provides salts, esters and prodrugs of the compounds of Formula I.

In certain embodiments, the compound of the application comprises the following structure:

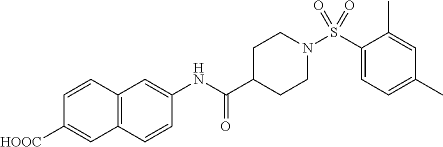 [SMILES: Cc1ccc(S(=O)(=O)N2CCC(C(=O)Nc3ccc4cc(C(=O)O)ccc4c3)CC2)c(C)c1]

Additionally, the present invention describes methods of synthesizing compounds of Formula I.

The present invention further provides a method of inhibiting the activity of soluble epoxide hydrolase (sEH), by contacting the sEH with a compound of Formula I in an amount effective to inhibit the activity of sEH.

In one embodiment, the sEH is expressed by a cell, for example, a mammalian cell, and the cell is contacted with the compound of Formula I.

In another embodiment, the sEH is contacted with the compound of Formula I in vitro.

The present invention also provides a method of decreasing the metabolism of an epoxyeicosatrienoic acid (EET), and thus increasing the level of an EET, by contacting an sEH with a compound of Formula I in an amount effective to increase the level of an EET.

The present invention also provides compositions comprising a compound of Formula I and a pharmaceutically acceptable carrier.

Also provided is a method for treating, preventing, or controlling diseases related to dysfunction of vasodilation, inflammation, and/or endothelial cells by administering to an individual in need of such treatment a pharmaceutical composition comprising a compound of Formula I in an amount effective to inhibit sEH activity or increase the level of EETs in the individual.

Also provided is a method for treating, preventing, or controlling metabolic syndrome by administering to an individual in need of such treatment a pharmaceutical composition comprising a compound of Formula I in an amount effective to inhibit sEH activity or increase the level of EETs in the individual.

### 4. BRIEF DESCRIPTION OF THE DRAWINGS

FIG. 1 Shows a reaction mechanism of a fluorescent high throughput screen encompassed by the present invention. In the screen, the sEH substrate PHOME fluoresces following sEH-catalyzed hydrolysis.

### 5. DETAILED DESCRIPTION

The present invention is based on the discovery of compounds that inhibit sEH enzymatic activity and increase the level of EETs in a cell. In light of the role EETs play in connection with vasodilation, inflammation, and endothelial cell function, the compounds of the instant invention can be used to increase EET levels and thereby ameliorate pathologies associated with diseases relating to vasodilation dysregulation, inflammation, and/or endothelial cell dysfunction.

For clarity and not by way of limitation, this detailed description is divided into the following sub-portions:

(i) definitions;

(ii) sEH inhibitors;

(iii) methods of treatment; and

(iv) pharmaceutical compositions.

### 5.1 DEFINITIONS

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

The terms “soluble epoxide hydrolase” and “sEH” refer to a polypeptide which catalyzes the addition of water to an epoxide substrate, resulting in a diol. In one non-limiting embodiment the epoxide substrate is a lipid epoxide. In another non-limiting embodiment, the substrate is an epoxyeicosatrienoic acid (EET).

In one non-limiting embodiment, a soluble epoxide hydrolase which may be inhibited according to the invention is a human soluble epoxide hydrolase. Such soluble epoxide hydrolase may, for example, be encoded by the human epoxide hydrolase 2, cytoplasmic gene (EPHX2) (GenBank accession number NM_{—}001979), a nucleic acid which encodes the human soluble epoxide hydrolase polypeptide. Alternatively, soluble epoxide hydrolase can be encoded by any nucleic acid molecule exhibiting at least 50%, at least 60%, at least 70%, at least 80%, at least 90% or up to 100% homology to the EPHX2 gene (as determined by standard software, e.g. BLAST or FASTA), and any sequences which hybridize under standard conditions to these sequences.

In other non-limiting embodiments, a soluble epoxide hydrolase which may be inhibited according to the invention may be characterized as having an amino acid sequence described by GenBank accession numbers: AAG14968, AAG14967, AAG14966 and NP_{—}001970, or any other amino acid sequence at least 90% homologous thereto.

The soluble epoxide hydrolase may be a recombinant sEH polypeptide encoded by a recombinant nucleic acid, for example, a recombinant DNA molecule, or may be of natural origin.

The terms “epoxyeicosatrienoic acid” and “EET” refer to a substrate of the soluble epoxide hydrolase enzyme. For example, an epoxyeicosatrienoic acid may have the following generic Formula II:

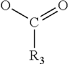

wherein R₃ is C₁₉H₃₁, and wherein an epoxide is bound to any two consecutive carbons of Formula II, and further, wherein any two consecutive carbons may be covalently bonded to each other by a double bond.

Substrate EETs, the cleavage of which are inhibited according to the invention, include effective regulators of blood pressure and cardiovascular function and/or inflammation.

In one such non-limiting embodiment, EET is an eicosanoid produced by the metabolic activity of a Cytochrome P450 epoxygenase on a fatty acid, such as arachidonic acid.

In another such non-limiting embodiment, the EET is a [5,6]-EET, as depicted in Formula III:

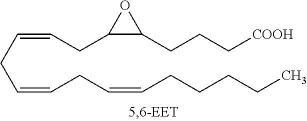 [SMILES: CCCCC/C=C\C/C=C\C/C=C\CC1OC1CCCC(=O)O]

In another such non-limiting embodiment, the EET is a [8,9]-EET, as depicted in Formula IV:

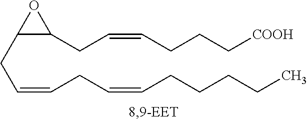 [SMILES: CCCCC/C=C\C/C=C\CC1OC1C/C=C\CCCC(=O)O]

In another such non-limiting embodiment, the EET is a [11,12]-EET, as depicted in Formula V:

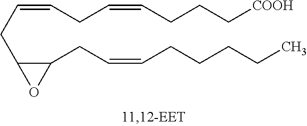 [SMILES: CCCCC/C=C\CC1OC1C/C=C\C/C=C\CCCC(=O)O]

In yet another such non-limiting embodiment, the EET is a [14,15]-EET, as depicted in Formula VI:

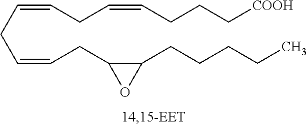 [SMILES: CCCCCC1OC1C/C=C\C/C=C\C/C=C\CCCC(=O)O]

In yet another non-limiting embodiment, the EET can function as a lipid mediator and can be incorporated into tissue phospholipids (Bernstrom et al. 1992, J. Biol. Chem. 267:3686-3690).

The term “dysfunction of vasodilation” refers to the reduced capability of a blood vessel, for example, an artery or arteriole, to dilate normally in response to an appropriate stimulus, for example, an endothelium derived hyperpolarizing factor, EDHF, and may be manifested by an inappropriate blood pressure, e.g. hypertension.

The term “endothelial cell dysfunction” refers to a physiological dysfunction of normal biochemical processes carried out by endothelial cells, the cells that line the inner surface of all blood vessels including arteries and veins. For example, endothelial cell dysfunction may result in an inability of blood vessels, such as arteries and arterioles, to dilate normally in response to an appropriate stimulus.

The term “inflammation” encompasses both acute responses (i.e., responses in which the inflammatory processes are active) as well as chronic responses (i.e., responses marked by slow progression and formation of new connective tissue).

In certain non-limiting embodiments, a disease associated with a dysfunction of vasodilation, inflammation, and/or endothelial cells that is to be treated by a compound of the instant invention is, by way of example, but not by way of limitation, heart disease, hypertension, such as primary or secondary hypertension, an ischemic condition such as angina, myocardial infarction, transient ischemic neurologic attack, cerebral ischemia, ischemic cerebral infarction, bowel infarction or other ischemic damage to tissue associated with poor perfusion.

In other non-limiting embodiments, a disease associated with inflammation that may be treated by a compound of the instant invention is, by way of example, but not by way of limitation, type I hypersensitivity, atopy, anaphylaxis, asthma, osteoarthritis, rheumatoid arthritis, septic arthritis, gout, juvenile idiopathic arthritis, still's disease, ankylosing spondylitis, inflammatory bowel disease, Crohn's disease or inflammation associated with vertebral disc herniation.

The term “metabolic syndrome” refers to risk factors that indicate an increased risk of developing coronary heart disease, type 2 diabetes and other diseases related to plaque buildups in artery walls, such as, for example, atherosclerosis, stroke and peripheral vascular disease. Metabolic syndrome risk factors include, for example, abdominal obesity (i.e. excessive fat tissue in and around the abdomen), atherogenic dyslipidemia (i.e. blood fat disorders such as for example, high triglycerides, low HDL cholesterol and high LDL cholesterol, that foster plaque buildups in artery walls), elevated blood pressure, insulin resistance or glucose intolerance, prothrombotic state (e.g., high fibrinogen or plasminogen activator inhibitor-1 in the blood) and/or a proinflammatory state (e.g., elevated C-reactive protein in the blood).

The term ‘alkyl’ refers to a straight or branched C₁-C₂₀ (preferably C₁-C₆) hydrocarbon group consisting solely of carbon and hydrogen atoms, containing no unsaturation, and which is attached to the rest of the molecule by a single bond, e.g., methyl, ethyl, n-propyl, 1-methylethyl(isopropyl), n-butyl, n-pentyl, 1,1-dimethylethyl(t-butyl).

The term “alkenyl” refers to a C₂-C₂₀ (preferably C₁-C₄) aliphatic hydrocarbon group containing at least one carbon-carbon double bond and which may be a straight or branched chain, e.g., ethenyl, 1-propenyl, 2-propenyl iso-propenyl, 2-methyl-1-propenyl, 1-butenyl, 2-butenyl.

The term “cycloalkyl” denotes an unsaturated, non-aromatic mono- or multicyclic hydrocarbon ring system (containing, for example, C₃-C₆) such as cyclopropyl, cyclobutyl, cyclopentyl, cyclohexyl. Examples of multicyclic cycloalkyl groups (containing, for example, C₆-C₁₅) include perhydronapththyl, adamantyl and norbornyl groups bridged cyclic group or sprirobicyclic groups, e.g., Spiro(4,4) non-2-yl.

The term “cycloalkalkyl” refers to a cycloalkyl as defined above directly attached to an alkyl group as defined above, that results in the creation of a stable structure such as cyclopropylmethyl, cyclobutylethyl, cyclopentylethyl.

The term “alkyl ether” refers to an alkyl group or cycloalkyl group as defined above having at least one oxygen incorporated into the alkyl chain, e.g., methyl ethyl ether, diethyl ether, tetrahydrofuran.

The term “alkyl amine” refers to an alkyl group or a cycloalkyl group as defined above having at least one nitrogen atom, e.g., n-butyl amine and tetrahydrooxazine.

The term “aryl” refers to aromatic radicals having in the range of about 6 to about 14 carbon atoms such as phenyl, naphthyl, tetrahydronapthyl, indanyl, biphenyl.

The term “arylalkyl” refers to an aryl group as defined above directly bonded to an alkyl group as defined above, e.g., —CH₂C₆H₅, and —C₂H₄C₆H₅.

The term “heterocyclic” refers to a stable 3- to 15-membered ring radical which consists of carbon atoms and one or more, for example, from one to five, heteroatoms selected from the group consisting of nitrogen, oxygen and sulfur. For purposes of this invention, the heterocyclic ring radical may be a monocyclic or bicyclic ring system, which may include fused or bridged ring systems, and the nitrogen, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heteroaromatic or heteroaryl aromatic).

The heterocyclic ring radical may be attached to the main structure at any heteroatom or carbon atom that results in the creation of a stable structure.

The term “heteroaryl” refers to a heterocyclic ring wherein the ring is aromatic.

The term “heteroarylalkyl” refers to heteroaryl ring radical as defined above directly bonded to alkyl group. The heteroarylalkyl radical may be attached to the main structure at any carbon atom from alkyl group that results in the creation of a stable structure.

The term “heterocyclyl” refers to a heterocylic ring radical as defined above. The heterocyclyl ring radical may be attached to the main structure at any heteroatom or carbon atom that results in the creation of a stable structure.

The term “halogen” refers to radicals of fluorine, chlorine, bromine and iodine.

### 5.2 SEH INHIBITORS

The present invention provides compounds of the following Formula I:

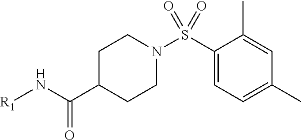

wherein R₁ is independently selected for each occurrence from the group consisting of substituted or unsubstituted alkyl, substituted or unsubstituted cycloalkyl, substituted or unsubstituted aryl, substituted or unsubstituted cycloalkalkyl, substituted or unsubstituted arylalkyl, substituted or unsubstituted heteroarylalkyl, substituted or unsubstituted heteroaryl, substituted or unsubstituted heterocyclic, substituted or unsubstituted alkoxy, substituted or unsubstituted aryloxy, phosphorous (e.g., substituted phosphorous such as diphenylphosphine), hydroxyl, hydrogen, substituted or unsubstituted ether, substituted or unsubstituted benzothiazol, substituted or unsubstituted pyridyl, substituted or unsubstituted naphthyl, substituted or unsubstituted phenyl, substituted or unsubstituted thienyl, substituted or unsubstituted benzothienyl, substituted or unsubstituted indol, substituted or unsubstituted isoquinolyl, substituted or unsubstituted quinolyl, —C(O)R² and —S(O)₂R², wherein R² is independently selected for each occurrence from the groups consisting of substituted or unsubstituted alkyl, substituted or unsubstituted cycloalkyl; substituted or unsubstituted aryl; substituted or unsubstituted arylalkyl; substituted or unsubstituted heteroaryl; substituted or unsubstituted heterocyclic, substituted or unsubstituted naphthyl, substituted or unsubstituted phenyl, substituted or unsubstituted thienyl, substituted or unsubstituted benzothienyl, substituted or unsubstituted pyridyl, substituted or unsubstituted indol, substituted or unsubstituted isoquinolyl, substituted or unsubstituted quinolyl, and substituted or unsubstituted benzothiazol.

The substituents in the substituted groups described herein, for example, ‘substituted or unsubstituted ether’, ‘substituted alkyl’, ‘substituted cycloalkyl’, ‘substituted cycloalkalkyl’, ‘substituted arylalkyl’, ‘substituted aryl’, ‘substituted heterocyclic’, ‘substituted heteroarylalkyl,’ ‘substituted heteroaryl’, ‘substituted naphthyl’, ‘substituted phenyl’, ‘substituted thienyl’, ‘substituted benzothienyl’, ‘substituted pyridyl’, ‘substituted indol’, ‘substituted isoquinolyl’, ‘substituted quinolyl’, or ‘substituted benzothiazol’ may be the same or different with one or more selected from the groups described in the present application and hydrogen, halogen, amide, acetyl, nitro, oxo (═O), thio —NO₂, —CF₃, —OCH₃, -Boc or optionally substituted groups selected from alkyl, alkoxy, aryl, aryloxy, arylalkyl, ether, ester, hydroxyl, heteroaryl, and heterocyclic ring. A “substituted” functionality may have one or more than one substituent.

In one non-limiting embodiment, R₁ is an unsubstituted cycloalkyl.

In other non-limiting embodiments, R₁ is an unsubstituted or substituted aryl having one or more substituent which is a halogen, more preferably fluorine or chlorine (where multiple substituents are present they may be the same or different).

In other non-limiting embodiments, R₁ is —S(O)₂R². In specific non-limiting embodiments R² is a substituted or unsubstituted aryl. In further specific non-limiting embodiments, R₁ is —S(O)₂R², where R² is a substituted aryl and the one or more substituent is selected from the group consisting of a hydrophobic alkyl group(s), such as the methyl group(s) present on toluene, xylene, and mesitylene, and a halide. In other non-limiting embodiments, at least one of said substituent of —S(O)₂R², where R² is a substituted aryl, is in the ortho position. In other non-limiting embodiments, the substituent of —S(O)₂R², where R² is a substituted aryl, is a bromide or fluoride or methyl at the ortho position.

In certain embodiments, the compound of the application comprises the following structure:

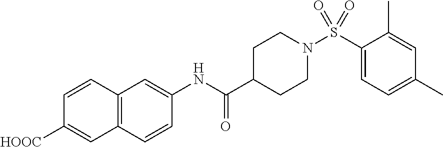 [SMILES: Cc1ccc(S(=O)(=O)N2CCC(C(=O)Nc3ccc4cc(C(=O)O)ccc4c3)CC2)c(C)c1]

Various non-limiting examples of compounds of the application are listed in Tables 1 and 2.

TABLE 1
Compounds of the Application

| | | Human | | |
|---|---|---|---|---|
| | | sEHIC50 | | |
| Structure | Inhibitor | (nM) | Origin | MolWeight |
| 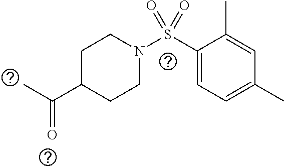 [SMILES: CC(=O)C1CCN(S(=O)(=O)c2ccc(C)cc2C)CC1] | 2534 | 100.000 | SP-II-4C | 297. [symbol] 7 |
| 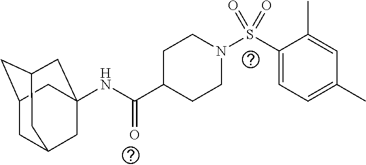 [SMILES: Cc1ccc(S(=O)(=O)N2CCC(C(=O)NC34CC5CC(CC(C5)C3)C4)CC2)c(C)c1] | 2535 | 8.5 | SP-II-5C | 430.60 [symbol] |
| 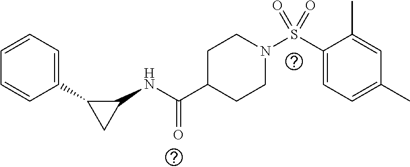 | 2536 | 5.2 | SP-II-6C | 412.545 |
| 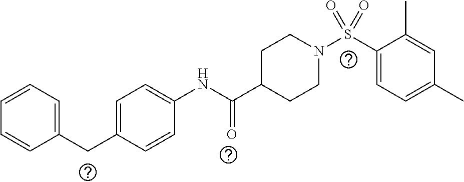 [SMILES: Cc1ccc(S(=O)(=O)N2CCC(C(=O)Nc3ccc(Cc4ccccc4)cc3)CC2)c(C)c1] | 2537 | 6 | SP-II-7C | 4 [symbol] .577 |
| 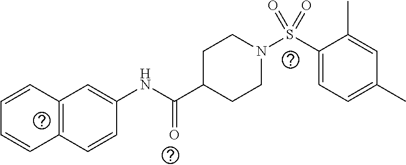 [SMILES: Cc1ccc(S(=O)(=O)N2CCC(C(=O)Nc3ccc4ccccc4c3)CC2)c(C)c1] | 2538 | 1. [symbol] | SP-II-8C | 422.54 |
| 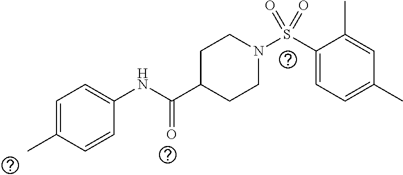 | 2539 | 6.9 | SP-II-10C | 440.479 |
| 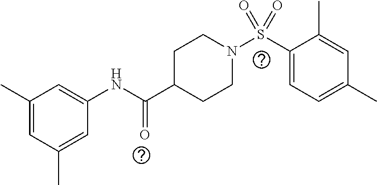 | 2540 | 12.3 | SP-II-14C | 441. [symbol] 71 |
| 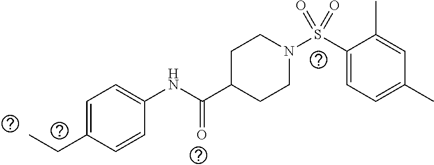 [SMILES: CCc1ccc(NC(=O)C2CCN(S(=O)(=O)c3ccc(C)cc3C)CC2)cc1] | 2541 | 5. [symbol] | SP-II-16C | 456.479 |
| 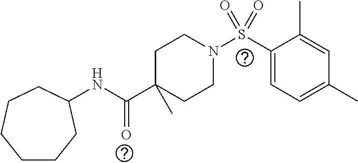 [SMILES: Cc1ccc(S(=O)(=O)N2CCC(C)(C(=O)NC3CCCCCC3)CC2)c(C)c1] | 2542 | 191.7 | SP-II-18C | 407.57 |
| 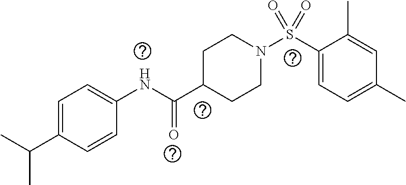 [SMILES: Cc1ccc(S(=O)(=O)N2CCC(C(=O)Nc3ccc(C(C)C)cc3)CC2)c(C)c1] | 2543 | [symbol] | SP-II-19C | 414.561 |
| 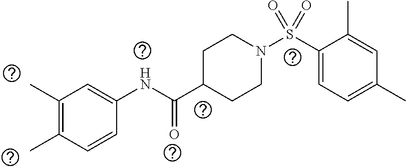 | 2544 | 6.7 | SP-II-20C | 441. [symbol] 1 |
| 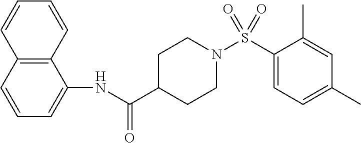 [SMILES: Cc1ccc(S(=O)(=O)N2CCC(C(=O)Nc3cccc4ccccc34)CC2)c(C)c1] | 2582 | 290. [symbol] | SP-II-48C | 423.528 |
| 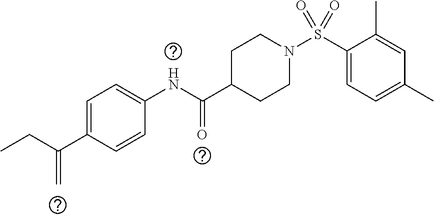 [SMILES: C=C(CC)c1ccc(NC(=O)C2CCN(S(=O)(=O)c3ccc(C)cc3C)CC2)cc1] | 2583 | 13.5 | SP-II-49C | 430.517 |
| 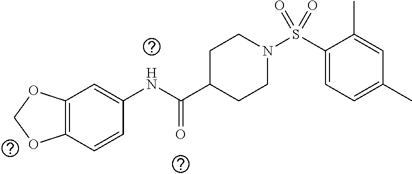 [SMILES: Cc1ccc(S(=O)(=O)N2CCC(C(=O)Nc3ccc4c(c3)OCO4)CC2)c(C)c1] | 2584 | 0. [symbol] | SP-II-50C | 416.4 [symbol] 1 |
| 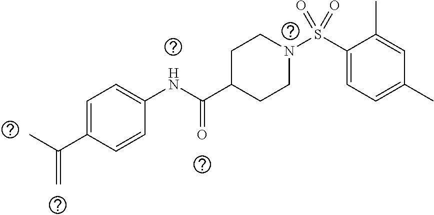 [SMILES: C=C(C)c1ccc(NC(=O)C2CCN(S(=O)(=O)c3ccc(C)cc3C)CC2)cc1] | 2585 | 108 | SP-II-51C | 416.4 [symbol] 1 |
| 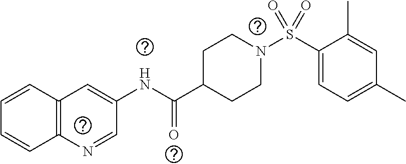 [SMILES: Cc1ccc(S(=O)(=O)N2CCC(C(=O)Nc3cnc4ccccc4c3)CC2)c(C)c1] | 2586 | 4 [symbol] .2 | SP-II-52C | 4 [symbol] .528 |
| 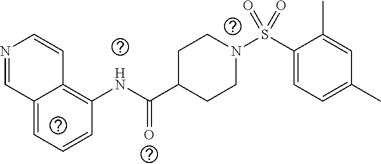 [SMILES: Cc1ccc(S(=O)(=O)N2CCC(C(=O)Nc3cccc4cnccc34)CC2)c(C)c1] | 2587 | 8. [symbol] | SP-II-53C | 423. [symbol] |
| 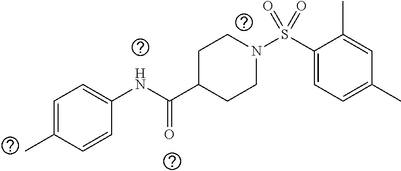 | 2588 | 4. [symbol] | SP-II-54C | 417.479 |
| 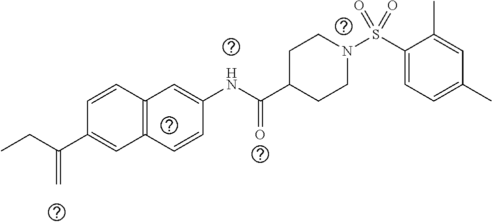 [SMILES: C=C(CC)c1ccc2cc(NC(=O)C3CCN(S(=O)(=O)c4ccc(C)cc4C)CC3)ccc2c1] | 2589 | 2. [symbol] | SP-II-55C | 480.57 [symbol] |
| 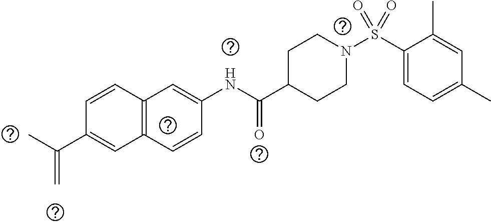 [SMILES: C=C(C)c1ccc2cc(NC(=O)C3CCN(S(=O)(=O)c4ccc(C)cc4C)CC3)ccc2c1] | 2590 | 23 .2 | SP-II-56C | 466.549 |
| 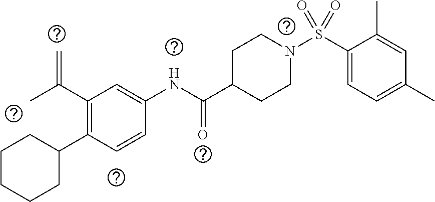 [SMILES: C=C(C)c1cc(NC(=O)C2CCN(S(=O)(=O)c3ccc(C)cc3C)CC2)ccc1C1CCCCC1] | 2591 | 101. [symbol] | SP-II-57C | 498.63 [symbol] |
| 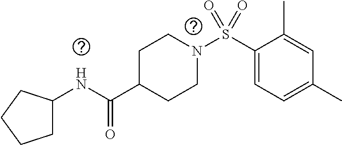 [SMILES: Cc1ccc(S(=O)(=O)N2CCC(C(=O)NC3CCCC3)CC2)c(C)c1] | 2592 | 6. [symbol] | SP-II-59C | 364.5 [symbol] |
| 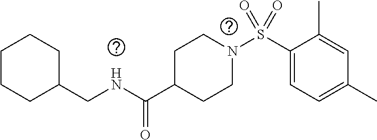 [SMILES: Cc1ccc(S(=O)(=O)N2CCC(C(=O)NCC3CCCCC3)CC2)c(C)c1] | 2593 | 1.2 | SP-II-59C | 392.55 [symbol] |
| 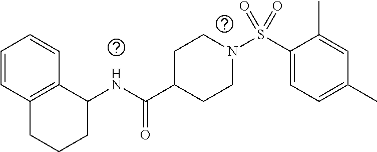 [SMILES: Cc1ccc(S(=O)(=O)N2CCC(C(=O)NC3CCCc4ccccc43)CC2)c(C)c1] | 2594 | 0. [symbol] | SP-II-60C | 426.572 |
| 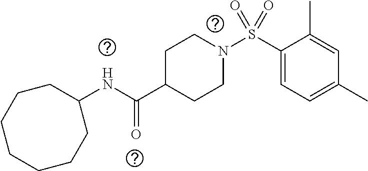 [SMILES: Cc1ccc(S(=O)(=O)N2CCC(C(=O)NC3CCCCCCC3)CC2)c(C)c1] | 2595 | 0.4 | SP-II-61C | 40 [symbol] .5 [symbol] |
| 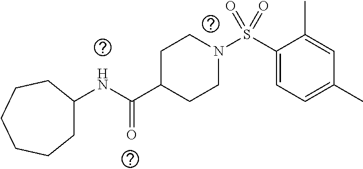 [SMILES: Cc1ccc(S(=O)(=O)N2CCC(C(=O)NC3CCCCCC3)CC2)c(C)c1] | 2596 | 0.4 | SP-II-95C | 392.55 [symbol] |
| 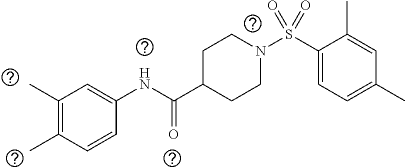 | 2562 | 48.3 | SP-II-22C | 474.924 |
| 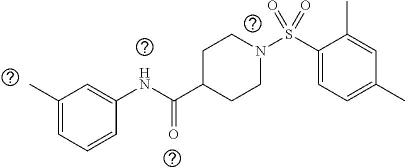 | 2563 | 19.4 | SP-II-23C | 440.479 |
| 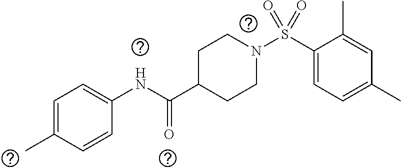 | 2564 | 16.9 | SP-II-25C | 451.377 |
| 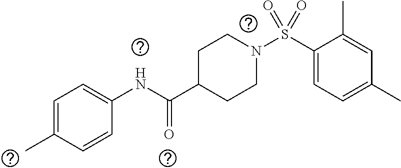 | 2565 | 19.5 | SP-II-26C | 406.925 |
| 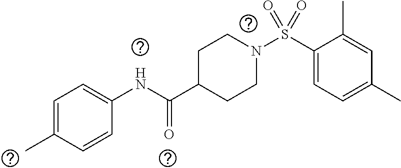 | 2566 | 25.5 | SP-II-27C | 390.472 |
| 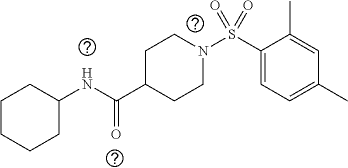 [SMILES: Cc1ccc(S(=O)(=O)N2CCC(C(=O)NC3CCCCC3)CC2)c(C)c1] | 2567 | 1.7 | SP-II-28C | 375.529 |
| 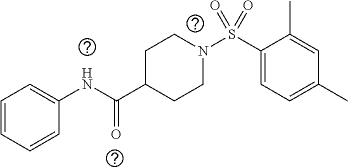 [SMILES: Cc1ccc(S(=O)(=O)N2CCC(C(=O)Nc3ccccc3)CC2)c(C)c1] | 2568 | 22.9 | SP-II-30C | 372.451 |
| 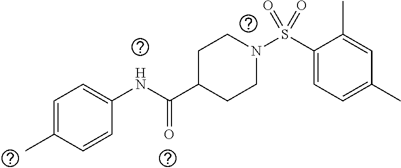 | 2569 | 54.5 | SP-II-32C | 386.491 |
| 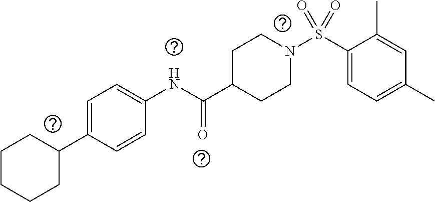 [SMILES: Cc1ccc(S(=O)(=O)N2CCC(C(=O)Nc3ccc(C4CCCCC4)cc3)CC2)c(C)c1] | 2570 | 29.2 | SP-II-35C | 455.613 |
| 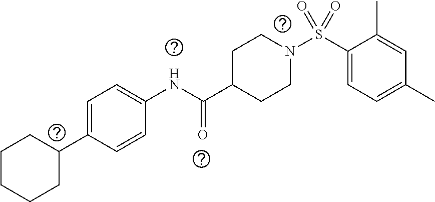 [SMILES: Cc1ccc(S(=O)(=O)N2CCC(C(=O)Nc3ccc(C4CCCCC4)cc3)CC2)c(C)c1] | 2570 | 41 | SP-II-40C | 457.598 |
| 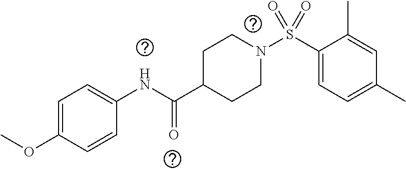 | 2571 | 25.6 | SP-II-41C | 402.507 |
| 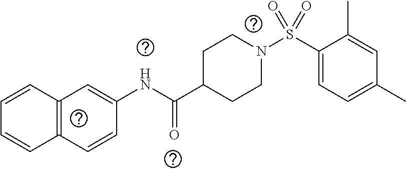 [SMILES: Cc1ccc(S(=O)(=O)N2CCC(C(=O)Nc3ccc4ccccc4c3)CC2)c(C)c1] | 2572 | 77.6 | SP-II-44C | 423.526 |

[symbol]

indicates data missing or illegible when filed

TABLE 2
Compounds of the Application

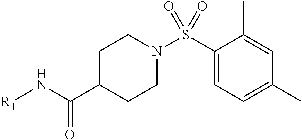

| Compound | R₁ | IC₅₀ ^{a, b}(nM) | Compound | R₁ | IC₅₀ (nM) |
|---|---|---|---|---|---|
| 7-1 | 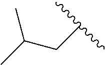 [SMILES: *CC(C)C] | 18 | 7-24 | 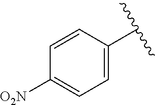 [SMILES: *c1ccc([N+](=O)[O-])cc1] | 4.6 |
| 7-2 | 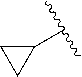 [SMILES: *C1CC1] | 6900 | 7-25 | 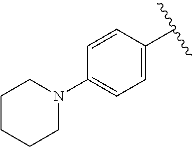 [SMILES: *c1ccc(N2CCCCC2)cc1] | 29 |
| 7-3 | 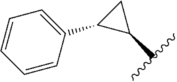 [SMILES: *[C@@H]1C[C@H]1c1ccccc1] | 5.2 | 7-26 | 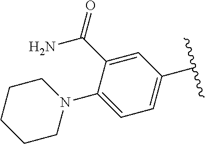 [SMILES: *c1ccc(N2CCCCC2)c(C(N)=O)c1] | 102 |
| 7-4 | 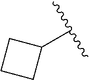 [SMILES: *C1CCC1] | 263 | 7-27 | 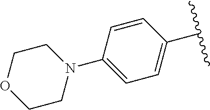 [SMILES: *c1ccc(N2CCOCC2)cc1] | 41 |
| 7-5 | 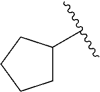 [SMILES: *C1CCCC1] | 5.8 | 7-28 | 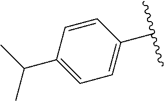 [SMILES: *c1ccc(C(C)C)cc1] | 2.8 |
| 7-6 | 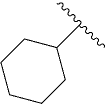 [SMILES: *C1CCCCC1] | 1.7 | 7-29 | 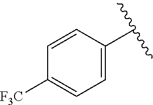 [SMILES: *c1ccc(C(F)(F)F)cc1] | 6.9 |
| 7-7 | 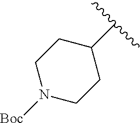 [SMILES: *C1CCN(C(=O)OC(C)(C)C)CC1] | 1.1 | 7-30 | 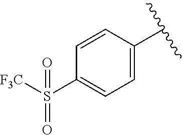 [SMILES: *c1ccc(S(=O)(=O)C(F)(F)F)cc1] | 8.7 |
| 7-8 | 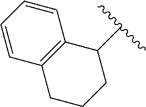 [SMILES: *C1CCCc2ccccc21] | 0.6 | 7-31 | 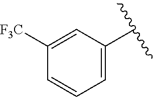 [SMILES: *c1cccc(C(F)(F)F)c1] | 20 |
| 7-9 | 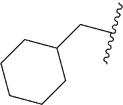 [SMILES: *CC1CCCCC1] | 1.2 | 7-32 | 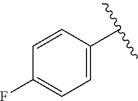 [SMILES: *c1ccc(F)cc1] | 25 |
| 7-10 | 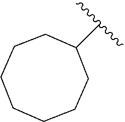 [SMILES: *C1CCCCCCC1] | 0.4 | 7-33 | 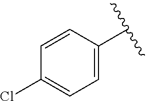 [SMILES: *c1ccc(Cl)cc1] | 20 |
| 7-11 | 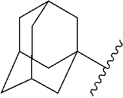 [SMILES: *C12CC3CC(CC(C3)C1)C2] | 8.5 | 7-34 | 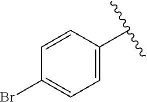 [SMILES: *c1ccc(Br)cc1] | 17 |
| 7-12 | 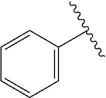 [SMILES: *c1ccccc1] | 23 | 7-35 | 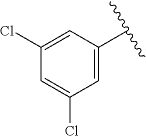 [SMILES: *c1cc(Cl)cc(Cl)c1] | 12 |
| 7-13 | 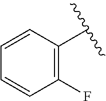 [SMILES: *c1ccccc1F] | 20 | 7-36 | 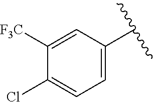 [SMILES: *c1ccc(Cl)c(C(F)(F)F)c1] | 43 |
| 7-14 | 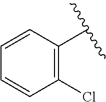 [SMILES: *c1ccccc1Cl] | 250 | 7-37 | 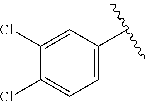 [SMILES: *c1ccc(Cl)c(Cl)c1] | 6.7 |
| 7-15 | 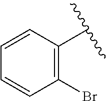 [SMILES: *c1ccccc1Br] | 30 | 7-38 | 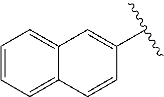 [SMILES: *c1ccc2ccccc2c1] | 1.6 |
| 7-16 | 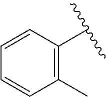 [SMILES: *c1ccccc1C] | 640 | 7-39 | 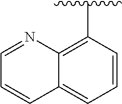 [SMILES: *c1cccc2cccnc12] | 290 |
| 7-17 | 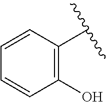 [SMILES: *c1ccccc1O] | 2200 | 7-40 | 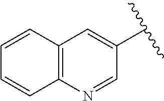 [SMILES: *c1cnc2ccccc2c1] | 45 |
| 7-18 | 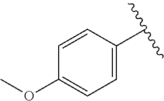 [SMILES: *c1ccc(OC)cc1] | 25 | 7-41 | 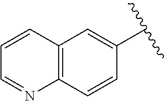 [SMILES: *c1ccc2ncccc2c1] | 78 |
| 7-19 | 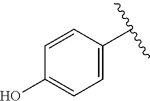 [SMILES: *c1ccc(O)cc1] | 55 | 7-42 | 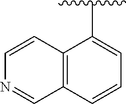 [SMILES: *c1cccc2cnccc12] | 8.3 |
| 7-20 | 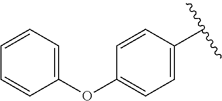 [SMILES: *c1ccc(Oc2ccccc2)cc1] | 6.0 | 7-43 | 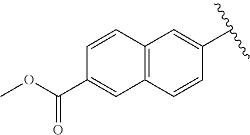 [SMILES: *c1ccc2cc(C(=O)OC)ccc2c1] | 2.3 |
| 7-21 | 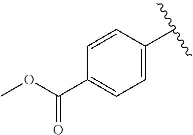 [SMILES: *c1ccc(C(=O)OC)cc1] | 13 | 7-44 | 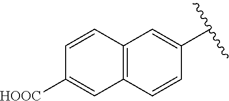 [SMILES: *c1ccc2cc(C(=O)O)ccc2c1] | 23 |
| 7-22 | 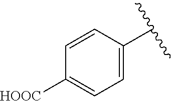 [SMILES: *c1ccc(C(=O)O)cc1] | 110 | 7-45 | 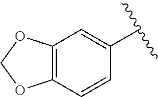 [SMILES: *c1ccc2c(c1)OCO2] | 0.6 |
| 7-23 | 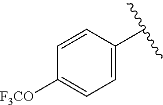 [SMILES: *c1ccc(OC(F)(F)F)cc1] | 5.2 | 7-46 | 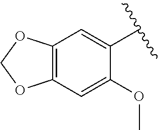 [SMILES: *c1cc2c(cc1OC)OCO2] | 30000 |

^{a}Reported IC₅₀ values are the average of three replicates. The fluorescent assay as performed here has a standard error between 10 and
20% suggesting that differences of two fold or greater are significant.¹
^{b}t-AUCB that has an IC₅₀ between 1 and 2 nM was used as positive control.²
References:
¹Jones, P. D.; Wolf, N. M.; Morisseau, C.; Whetstone, P.; Hock, B.; Hammock, B. D. Anal. Biochem. 2005, 343, 66.
²Hwang, S. H.; Tsai, H. J.; Liu, J. Y.; Morisseau, C.; Hammock, B. D. J. Med. Chem. 2007, 50, 3825.

Compounds of Formula I may, without limitation, be synthesized by any means known in the art. For example, a sulfonamide can be prepared from methyl isonipecotate and 2,4-dimethylbenzenesulfonyl chloride. Saponification of the methyl ester sulfonamide. with, for example, LiOH, produces an acid form of the compound. EDC peptide coupling reactions of the acid compound with various amines to produce compounds of Formula I.

In other non-limiting embodiments, the compounds of Formula I may be synthesized according to the following scheme:

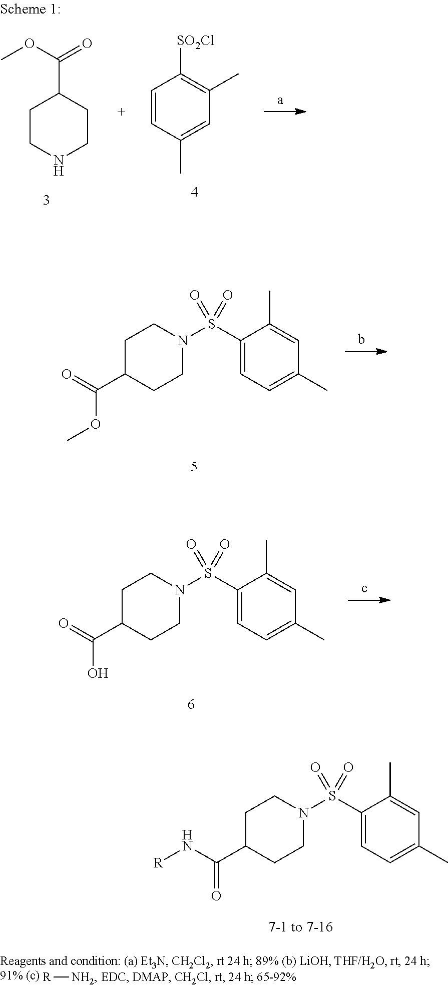

wherein R₁ is selected from the compounds described previously for Formula I.

In other non-limiting embodiments, compounds of Formula I may be synthesized, for example, by protecting methyl isonipecotate with benzyl chloroformate, and then converting the compound into an acid chloride by removing the methyl ester followed by treatment with oxalyl chloride. Coupling of the acid chloride with a reactive amine substituent of the present application (i.e., an R₁ reactive amine), for example, 2,4-dichlorobenzylamine, followed by Palladium catalyzed hydrogenation produces an amine, which may be reacted with sulfonyl chloride, to produce compounds of Formula I.

In other non-limiting embodiments, methyl isonipecotate may be treated with xylenesulfonyl chloride followed by conversion into acid chloride by removing the methyl ester followed by treatment with oxalyl chloride. The acid chloride may then be reacted with various amines to produce compounds of Formula I.

In other non-limiting embodiments, the compounds of Formula I may be synthesized according to the following scheme:

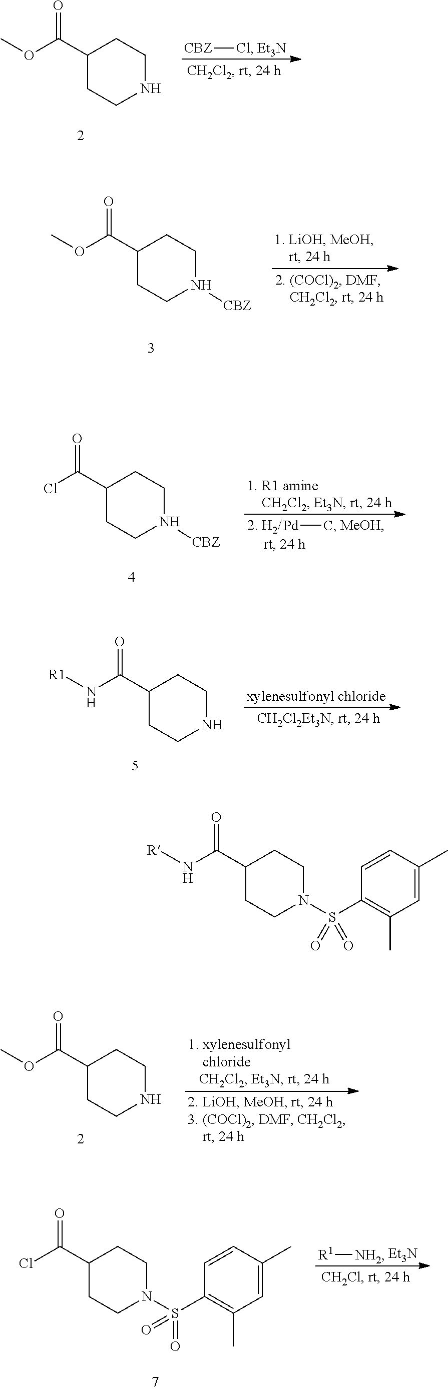

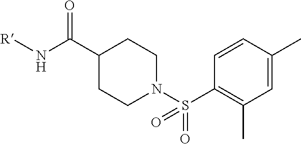

wherein R₁ is selected from the compounds described previously for Formula I.

### 5.3 METHODS OF TREATMENT

In accordance with the invention, there are provided methods of using the compounds of Formula I. The compounds used in the invention may be used to inhibit the degradation of sEH substrates having beneficial effects and/or inhibit the formation of metabolites that have adverse effects. The methods of the invention may be used to treat a variety of diseases related to dysfunction of vasodilation, inflammation, and/or endothelial cells. For example, the methods of the invention are useful for the treatment of conditions including, but not limited to, hypertension, such as primary or secondary hypertension, ischemic conditions such as angina, myocardial infarction, transient ischemic neurologic attack, cerebral ischemia, ischemic cerebral infarction, bowel infarction, etc. Additionally, inflammatory conditions including, but not limited to, type I hypersensitivity, atopy, anaphylaxis, asthma, osteoarthritis, rheumatoid arthritis, septic arthritis, gout, juvenile idiopathic arthritis, still's disease, ankylosing spondylitis, inflammatory bowel disease, Crohn's disease or inflammation associated with vertebral disc herniation may be treated according to the methods of the present invention. The invention may also be used to reduce the risk of ischemic damage to tissue associated with atherosclerosis.

In certain non-limiting embodiments, the compounds of Formula I used in the methods of treatment described herein are the compounds described in Table 1, Table 2 or Table 3 of the present application.

In certain non-limiting embodiments, the compounds of Formula I used in the methods of treatment described herein are compounds 2, 7-3, 7-6, 7-9, 7-11, 7-20, 7-23, 7-24, 7-37, 7-38, 7-42, 7-44 or 7-45.

In certain non-limiting embodiments, one or more of the compounds of Formula I described herein can be used in the methods of the present application.

According to the invention, a “subject” or “patient” is a human or non-human animal. Although the animal subject is preferably a human, the compounds and compositions of the invention have application in veterinary medicine as well, e.g., for the treatment of domesticated species such as canine, feline, and various other pets; farm animal species such as bovine, equine, ovine, caprine, porcine, etc.; wild animals, e.g., in the wild or in a zoological garden; and avian species, such as chickens, turkeys, quail, songbirds, etc.

In one embodiment, the subject or patient has been diagnosed with, or has been identified as having an increased risk of developing, a disease related to dysfunction of vasodilation, inflammation, and/or an endothelial cell dysfunction.

In other non-limiting embodiments, the present invention provides for methods of reducing the risk of damage resulting from diseases related to dysfunction of vasodilation, inflammation, and/or endothelial cell dysfunction to a tissue of a subject comprising administering to the subject, an effective amount of a composition according to the invention.

The present invention provides for methods of treating diseases related to dysfunction of vasodilation, inflammation, and/or endothelial cell dysfunction in a subject in need of such treatment by administration of a therapeutic formulation which comprises a compound of Formula I. In particular embodiments, the formulation may be administered to a subject in need of such treatment in an amount effective to inhibit sEH enzymatic activity. Where the formulation is to be administered to a subject in vivo, the formulation may be administered systemically (e.g. by intravenous injection, oral administration, inhalation, etc.), or may be administered by any other means known in the art. The amount of the formulation to be administered may be determined using methods known in the art, for example, by performing dose response studies in one or more model system, followed by approved clinical testing in humans.

In another non-limiting embodiment of the invention, a subject to be treated with a compound of Formula I suffers from metabolic syndrome, wherein administering a compound of Formula I to the subject reduces the subject's risk of developing coronary heart disease, type 2 diabetes and other diseases related to plaque buildups in artery walls, such as, for example, atherosclerosis, stroke and peripheral vascular disease.

In another non-limiting embodiment, the invention provides a method for inhibiting the activity of a soluble epoxide hydrolase which comprises contacting the soluble epoxide hydrolase with a compound of Formula I in an amount effective to inhibit soluble epoxide hydrolase activity.

In other non-limiting embodiments, the invention provides a method for treating a disease related to dysfunction of vasodilation, inflammation, and/or endothelial cell dysfunction in an individual, which method comprises administering to the individual an effective amount of a compound according to Formula I.

In certain non-limiting embodiments of the invention, an effective amount of compound is an amount which results in a blood level of compound which is at least 20% or at least 50% or at least 90% of the IC₅₀. Non-limiting specific examples of compounds of the invention and their IC₅₀ values are shown in Tables 1 and 2.

According to the invention, an effective amount is an amount of a compound of Formula I which reduces the clinical symptoms of diseases related to dysfunction of vasodilation, inflammation, and/or endothelial cells. For example, an effective amount is an amount of a compound of Formula I that reduces abnormally high arterial blood pressure (for example but not by way of limitation, abnormally high systolic pressure, diastolic pressure, or both, wherein systolic blood pressure is at least 140 mm Hg and a diastolic blood pressure is at least 90 mm Hg), or inflammation in a subject, or increases the flow of blood to an organ or tissue, for example but not by way of limitation, the heart or brain in a subject.

In a further non-limiting embodiment, the effective amount of a compound of Formula I may be determined via an in vitro assay. By way of example, and not of limitation, such an assay may utilize an sEH enzyme and a substrate which can report the level of sEH activity through a detectable signal, such as, for example, a change in luminescence, coloration, temperature, or fluorescence. In one embodiment, the assay is a high throughput fluorescent assay that utilizes a recombinant human sEH and a water soluble α-cyanocarobonate epoxide (PHOME) substrate (see, e.g., Wolf et al., 2006, Anal. Biochem 335:71-80). According to the invention, the assay can be initiated by sEH-catalyzed hydrolysis of the non-fluorescent PHOME substrate followed by spontaneous cyclization to give a cyanohydrin. Under basic condition, the cyanohydrin rapidly decomposes into a highly fluorescent product. Fluorescence with excitation at 320 nm and emission at 460 nm can be recorded at the endpoint of the reaction cascade with or without the presence of assay samples. When the hydrolysis reaction is performed in the presence of a compound of Formula I, a decrease in recorded fluorescence indicates inhibition of sEH enzymatic activity, wherein a greater decrease in fluorescence indicates a greater inhibition of sEH.

In one non-limiting embodiment, an effective amount of a compound of Formula I may be an amount that results in a local concentration of compound at the therapeutic site (such as, but not limited to, the serum concentration) of from at least about 0.01 nM to about 2 uM, or from at least about 0.01 nM to about 200 nM, or from at least about 0.01 nM to about 50 nM.

In another non-limiting embodiment, an effective amount of a compound of Formula I may be correlated with the compound's ability to inhibit sEH activity by at least about 5-10%, or from at least about 10-20%, or from at least about 20-30%, or from at least about 30-40%, or from at least about 40-50%, or from at least about 50-60%, or from at least about 60-70%, or from at least about 70-80%, or from at least about 80-90%, or from at least about 90-100%, when the compound is administered in the in vitro assay, wherein a greater level of sEH inhibition at a lower concentration in the in vitro assay is correlative with the compound's therapeutic efficacy.

In a further non-limiting embodiment, the compound is administered at a concentration of 200 nM in the in vitro assay.

In a non-limiting embodiment, an effective amount of a compound of Formula I may be correlated with the compound's ability to inhibit sEH activity by about at least 60% when the compound is administered at a concentration of 200 nM in the in vitro assay.

In other non-limiting embodiments, an effective amount of a compound of Formula I may be correlated with the compound's ability to inhibit sEH activity by about at least 70% when the compound is administered at a concentration of 200 nM in the in vitro assay.

In other non-limiting embodiments, an effective amount of a compound of Formula I may be correlated with the compound's ability to inhibit sEH activity by about at least 80% when the compound is administered at a concentration of 200 nM in the in vitro assay.

In other non-limiting embodiments, an effective amount of a compound of Formula I may be correlated with the compound's ability to inhibit sEH activity by about at least 90% when the compound is administered at a concentration of 200 nM in the in vitro assay.

In other non-limiting embodiments, an effective amount of a compound of Formula I may be correlated with the compound's ability to inhibit sEH activity by about at least 95% when the compound is administered at a concentration of 200 nM in the in vitro assay.

In other non-limiting embodiments, an effective amount of a compound of Formula I may be correlated with the compound's ability to inhibit sEH activity by about 100% when the compound is administered at a concentration of 200 nM in the in vitro assay.

In another non-limiting embodiment, an effective amount of a compound of Formula I may be correlated with the compound's ability to inhibit sEH activity by at least about 50% compared to a control cell line that was not contacted with the candidate compound (i.e., IC₅₀), wherein the compound is tested at a concentration ranging from at least about 200 nM to about 0.01 nM, or from at least about 100 nM to about 0.01 nM, or from at least about 10 nM to about 0.01 nM in the in vitro assay, wherein such inhibition of sEH activity at the above-described concentrations is correlative with the compound's therapeutic efficacy.

In other non-limiting embodiments, an effective amount of a compound of Formula I may be correlated with the compound's ability to inhibit sEH activity by about at least 50% when the compound is administered at a concentration of about 90 nM in the in vitro assay.

In other non-limiting embodiments, an effective amount of a compound of Formula I may be correlated with the compound's ability to inhibit sEH activity by about at least 50% when the compound is administered at a concentration of 80 nM in the in vitro assay.

In other non-limiting embodiments, an effective amount of a compound of Formula I may be correlated with the compound's ability to inhibit sEH activity by about at least 50% when the compound is administered at a concentration of about 40 nM in the in vitro assay.

In other non-limiting embodiments, an effective amount of a compound of Formula I may be correlated with the compound's ability to inhibit sEH activity by about at least 50% when the compound is administered at a concentration of about 20 nM in the in vitro assay.

In other non-limiting embodiments, an effective amount of a compound of Formula I may be correlated with the compound's ability to inhibit sEH activity by about at least 50% when the compound is administered at a concentration of about 23 nM in the in vitro assay.

In other non-limiting embodiments, an effective amount of a compound of Formula I may be correlated with the compound's ability to inhibit sEH activity by about at least 50% when the compound is administered at a concentration of about 10 nM in the in vitro assay.

In other non-limiting embodiments, an effective amount of a compound of Formula I may be correlated with the compound's ability to inhibit sEH activity by about at least 50% when the compound is administered at a concentration of about 5 nM in the in vitro assay.

In other non-limiting embodiments, an effective amount of a compound of Formula I may be correlated with the compound's ability to inhibit or reduce inflammation or pain, for example, mechanical allodynia or thermal hyperalgesia, in vivo, wherein a greater reduction in inflammation or pain at a lower concentration compared to a control subject that is not administered the compound is correlative with the compound's therapeutic efficacy. By way of example, and not of limitation, such an in vivo assay may comprise administering a compound of Formula I to a test subject, for example, a mouse or rat, followed by an assay to determine a change in inflammation or pain in the subject. The assay used to measure inflammation or pain may be any assay known in the art, for example, behavioral assays such as an electronic Von Frey test, tail flick assay or thermal paw withdrawal test.

In one embodiment, inflammation or pain may be induced in the subject using methods known in the art, such as, for example, by administering Complete Freund's Adjuvant (CFA) to the test subject. The inflammation or pain may be induced prior to, at the same time as, or after administration of the compound of Formula I. When inflammation or pain is induced before the administration of a compound of Formula I, the inflammation or pain may be induced at least 5 minutes, at least 30 minutes, at least 1 hour, at least 5 hours, at least 10 hours, at least 24 hours, at least 2 days, at least 5 days, or at least 1 week or more before the compound of Formula I is administered. The level of inflammation or pain in the test subject may be assayed following induction.

In another embodiment of the invention, the level of inflammation or pain in the test subject may be assayed before inflammation or pain is induced. Inflammation or pain may be assayed again when the compound of Formula I is administered, and at intervals following administration of the compound, for example, at intervals of at least 5 seconds, at least 10 seconds, at least 30 seconds, at least 1 minute, at least 5 minutes, at least 30 minutes, at least 1 hour, at least 5 hours, at least 10 hours, at least 24 hours, at least 2 days, at least 5 days, or at least 1 week, or combinations thereof, following administration of the compound.

According to the invention, the component or components of a pharmaceutical composition of the invention may be introduced by intravenous, intra-arteriole, intramuscular, intradermal, transdermal, subcutaneous, oral, intraperitoneal, intraventricular, and intrathecal administration.

In yet another embodiment, the therapeutic compound can be delivered in a controlled or sustained release system. For example, a compound or composition may be administered using intravenous infusion, an implantable osmotic pump, a transdermal patch, liposomes, or other modes of administration. In one embodiment, a pump may be used (see Sefton, 1987, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used (see Langer and Wise eds., 1974, Medical Applications of Controlled Release, CRC Press: Boca Raton, Fla.; Smolen and Ball eds., 1984, Controlled Drug Bioavailability, Drug Product Design and Performance, Wiley, N.Y.; Ranger and Peppas, 1983, J. Macromol. Sci. Rev. Macromol. Chem., 23:61; Levy et al., 1985, Science 228:190; During et al., 1989, Ann. Neurol., 25:351; Howard et al., 9189, J. Neurosurg. 71:105). In yet another embodiment, a controlled release system can be placed in proximity of the therapeutic target, i.e., the heart or a blood vessel, thus requiring only a fraction of the systemic dose (see, e.g., Goodson, 1984, in Medical Applications of Controlled Release, supra, Vol. 2, pp. 115-138). Other controlled release systems known in the art may also be used.

### 5.4 PHARMACEUTICAL COMPOSITIONS

The compounds and compositions of the invention may be formulated as pharmaceutical compositions by admixture with a pharmaceutically acceptable carrier or excipient.

In one non-limiting embodiment, the pharmaceutical composition may comprise an effective amount of a compound of Formula I and a physiologically acceptable diluent or carrier. The pharmaceutical composition may further comprise a second drug, for example, but not by way of limitation, an anti-hypertension drug or an anti-inflammatory drug.

The phrase “pharmaceutically acceptable” refers to molecular entities and compositions that are physiologically tolerable when administered to a subject. Preferably, but not by way of limitation, as used herein, the term “pharmaceutically acceptable” means approved by a regulatory agency of the federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, or, for solid dosage forms, may be standard tabletting excipients. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition, or other editions.

In a specific embodiment, the therapeutic compound can be delivered in a vesicle, in particular a liposome (see Langer, 1990, Science 249:1527-1533; Treat et al., 1989, in Liposomes in the Therapy of Infectious Disease and Cancer, Lopez-Berestein and Fidler eds., Liss: New York, pp. 353-365; Lopez-Berestein, ibid., pp. 317-327; see generally Lopez-Berestein,

### 6. EXAMPLES

### Example 1

### Screening Assay to Identify Inhibitors of sEH

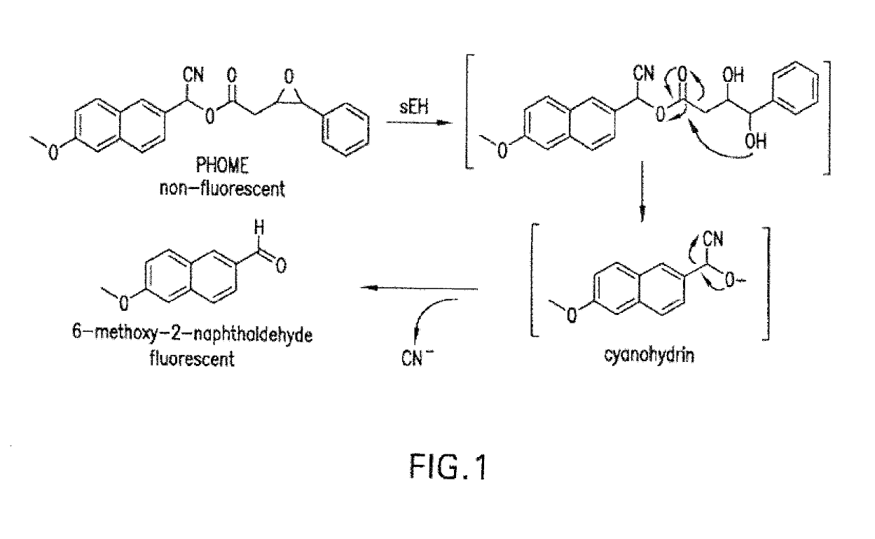

A fluorescent assay was employed for high throughput screening (HTS) of inhibitors of sEH. This HTS employs recombinant human sEH and a water soluble α-cyanocarobonate epoxide (PHOME) as the substrate (Wolf et. al. Anal Biochem. 2006, 335, 71). As shown in FIG. 1, the assay was initiated by sEH-catalyzed hydrolysis of the non-fluorescent substrate followed by spontaneous cyclization to give a cyanohydrin. Under basic condition, the cyanohydrin rapidly decomposed into a highly fluorescent product. Fluorescence with excitation at 320 nm and emission at 460 nm was recorded at the endpoint of the reaction cascade with or without the presence of assay samples.

A library of compounds was created for screening using the assay described above. The library was assembled according to the following synthesis. Sulfonamide was prepared from methyl isonipecotate and 2,4-dimethylbenzenesulfonyl chloride (Sigma Aldrich, St. Louis, Mo.). Saponification of this methyl ester with LiOH afforded an acid compound. EDC peptide coupling reactions of the acid compound with various commercially available amines provided the compounds of the invention.

New compounds were first screened at concentrations of 2 μM, 400 nm and 200 nm using the fluorescence assay described above. The IC₅₀s were further determined for those compounds showing more than 50% inhibition at the concentration of 200 nm. The biological results for the modification are summarized in Table 1.

### Example 2

### Screening Assay to Identify Inhibitors of sEH

A fluorescent assay was employed for high throughput screening (HTS) of inhibitors of sEH. Cyano(2-methoxynaphthalen-6-yl)methyl trans-(3-phenyloxyran-2-yl)methyl carbonate (CMNPC) was used as the fluorescent substrate. Human sEH (1 nM) was incubated with a compound of Formula I for 5 min in pH 7.0 Bis-Tris/HCl buffer (25 mM) containing 0.1 mg/mL of BSA at 30° C. prior to substrate introduction ([S]=5 μM). Activity was determined by monitoring the appearance of 6-methoxy-2-naphthaldehyde over 10 min by fluorescence detection with an excitation wavelength of 330 nm and an emission wavelength of 465 nm. IC₅₀ values are the average of the three replicates with at least two datum points above and at least two below the IC₅₀.

A library of compounds was created for screening using the assay described above. The library was assembled according to the following synthesis. A sulfonamide was prepared from methyl isonipecotate and 2,4-dimethylbenzenesulfonyl chloride (Sigma Aldrich, St. Louis, Mo.). Saponification of this methyl ester with LiOH afforded an acid compound. EDC peptide coupling reactions of the acid compound with various commercially available amines provided the compounds of the application. The IC₅₀ values for the compounds of Formula I tested are shown in Table 2, described above.

Several sEH inhibitors were identified possessing improved or similar potency compared to lead compound 2596, specifically compound 7-10 showed an IC₅₀ of 0.4 nM, the most potent amide non-urea sEH inhibitor reported to date. Replacement of cycloalkyl ring with a more compact phenyl ring (compound 7-12), resulted in 15-fold drop in potency against human sEH. Introduction of the phenyl ring allowed access to electronically and sterically diverse structures, and attachment of various polar groups. Placement of fluorine or bromine in the ortho position did not significantly change the potency of the non-urea inhibitors (7-13 and 7-15), while chlorine and methyl group decreased the potency for 10 and 30-fold, respectively (7-14 and 7-16). Polar hydroxyl group in ortho position showed a negative effect on potency in non-urea based compounds (7-17). Although the para substitution is generally tolerated, placement of polar substituents resulted in less potent inhibitors.

Placement of methoxy group in para position (compound 7-18) did not significantly changed the potency compared to compound 7-12, while introduction of hydroxyl group in the same position (compound 7-19 can be observed as a metabolite of 7-18) led to a two fold decreased potency. Similar results were observed for methyl ester compound 7-21 and its corresponding carboxylic acid compound 7-22. The 4-trifluoromethoxyphenyl analog 7-23 was synthesized. A fourfold increase in potency was observed for this compound compared with compound 7-12. 7-23 was selected for further pharmacokinetic studies. The analog 7-24, showed a five fold increase in activity comparing to phenyl compound 7-12, despite the presence of the high polarity nitro functionality. The metabolic stability for this inhibitor was evaluated as well. A basic nitrogen was introduced (piperidine and morpholine rings in para position; analogs 7-25, 7-26 and 7-27) in order to allow formulation of the inhibitor as a salt. These modifications did not improve the potency, similar to other polar substituents in this position. On the other hand, the inhibition potencies increased when small non-polar para or meta substituents were added (7-28, 7-29, 7-30 and 7-31). Since halogens can enhance polarity and decrease the rate of metabolism degradation due to their electron withdrawing effect on the aromatic ring, a set of analogs containing various halogens in different position on the left-hand side phenyl moiety were prepared. The fluorinated, chlorinated and brominated para-phenyl compounds (7-32, 7-33 and 7-34, respectively) did not show significant improvement in activity compared to compound 7-12. Placement of two chlorine atoms in meta, and meta and para positions showed a twofold and threefold lower IC₅₀ against human sEH enzyme, 7-35 and 7-37, respectively.

Inclusion of 2-naphthalene on the left side of the molecule 7-38 resulted in high potency against the human enzyme, which is already shown in recent literature (Rose et al., J. Med. Chem. 2010, 53, 7067). Thus, in vitro metabolic profile for this compound was tested. A nitrogen was introduced in this moiety in order to improve physical properties and for the ease of formulation. Various amino quinolines were attached via different position to the central non-urea moiety. 5-Aminoquinoline derivative 7-39 led to five fold lower potency against sEH, 3-aminoquinoline derivative 7-40 showed 30-fold diminished potency, while 6- and 8-aminoquinoline analogs 7-41 and 7-42, led to even more drastically decreased potency, 50-fold and 180-fold, respectively.

Polar groups were next introduced into position 6 of the 2-naphthalene moiety. Methylester analog 7-43 showed slight decrease in activity, while corresponding carboxylic acid 7-44 had 15-folds lower inhibition then the 2-naphthalene analog. 3,4-methylenedioxybenzene analog 7-45 resulted in a subnano molar potent inhibitor of human sEH enzyme. Selected non-urea sEH inhibitors were profiled in a human liver microsomal assay (Example 4) as a predictor of in vivo oxidative metabolism (Table 3).

The present study describes the structure-activity relationship of particular modifications to the structure of the left-hand side part of the piperidine amide-based sEH inhibitor compound 2596. A varying degree of bulky, nonpolar cycloalkyl rings are well tolerated in this region by target enzyme. In contrast, proper substitution on the phenyl ring is crucial for attaining good potency, emphasizing the importance of the small nonpolar groups and halogens in the para position as a recognition element for sEH, suggesting that left-hand side phenyl is in a relatively close proximity to a several hydrophobic residues located in the large, non-polar pocket of sEH that opens towards solvent, and may participate in a p-stacking interaction with them.

### Example 3

### In Vivo Effect of sEH Inhibitors on Mechanical Allodynia and Thermal Hyperalgesia

The effectiveness of a compound of Formula I in reducing pain sensitivity can be examined in vivo. Inflammation can be induced by injection of Complete Freund's Adjuvant (CFA) into the footpad of mice at day 1 of the study. 24 hours following CFA administration, two test animals can be administered a subcutaneous injection of a compound of Formula I. The compound can be dissolved in 100% DMSO prior to administration. As a positive control, an analgesic effect can be elicited in one animal by administering the Protein Kinase G (PKG) inhibitor RPG (exemplary RPGs include Rp-cGMPs) intrathecally 24 hours after CFA administration. As a negative control, one animal can be administered an intrathecal injection of saline and a subcutaneous injection of 100% DMSO 24 hours after CFA. Additionally, animals can be administered a subcutaneous injection of a compound of Formula I and an intrathecal injection of RPG 24 hours after CFA to determine if the two compounds can achieve an additive or synergistic analgesic effect.

Pain sensitivity can be measured using behavioral assays. The electronic Von Frey test can be used to measure mechanical allodynia in the control and test animals, while the thermal paw withdrawal test can be used to measure thermal hyperalgesia. The electronic Von Frey test consists of application of a filament against the rodent's paw, whereby paw withdrawal caused by the stimulation is registered as a response. The corresponding force (resistance) applied can be recorded in grams. The thermal paw withdrawal test comprises applying a thermal stimulus to the rodent's foot, whereby the withdrawal latency can be measured as a response.

A baseline sensitivity to pain can be first measure prior to CFA treatment, and again after administration of CFA. Pain sensitivity can then be assayed 24 hours later at day 2 following the administration of the compounds of Formula I or the control agents, and again at day 5.

### Example 4

### In Vitro Human Liver Microsomal Metabolic Stability of sEH Inhibitors

The stability of sEH inhibitors in a human liver microsomal assay was determined as a predictor of in vivo oxidative metabolism. Microsomal stability was assessed in pooled human liver microsomes (Celsis, Edison, N.J.). All reactions were carried out for 90 min at 37° C. in an NADPH-generating system consisting of glucose 6-phosphate, glucose 6-phosphate dehydrogenase, and NADP⁺ (Sigma, St. Louis, Mo.). Positive control incubations proceeded with 7-ethoxycoumarin as the substrate. Reactions were terminated by adding methanol. The mixtures were centrifuged and the supernatants were evaporated. The residues were reconstituted in mobile phase (85% ACN; 15% H₂O) and subjected to LC/MS analysis.

The results from this assay are shown in Table 3. The results show that compounds tested with aromatic moiety substituent R groups exhibited a better metabolic profile in the human liver microsomal assay than compounds tested with hydrophobic cycloalkyl substituent R groups, such as cyclohexyl, methylcyclohexyl, cycloheptyl, cyclooctyl or adamantyl. Evaluation of the in vitro metabolic stability of aromatic compounds revealed intermediate metabolic profiles for compounds with para-substitution (compounds 7-25 and 7-26), with the exception of the carboxylic acid derivative 7-44, which demonstrated excellent in vitro metabolic stability in human liver microsomes.

TABLE 3

| | hLM t_{1/2} | CLᵢₙₜ, ₐₚₚ |
|---|---|---|
| Compound | (min)^{a} | (mL/min/kg)^{b} |
| 2 | 5.5 | 220 |
| 7-3 | 14 | 90 |
| 7-6 | 14 | 90 |
| 7-9 | 2.4 | 520 |
| 7-11 | 3.7 | 340 |
| 7-20 | 11 | 120 |
| 7-23 | 46 | 28 |
| 7-24 | 180 | 7.0 |
| 7-37 | 25 | 50 |
| 7-38 | 36 | 35 |
| 7-42 | 8.7 | 140 |
| 7-44 | 220 | 5.6 |
| 7-45 | 36 | 35 |

^{a}Data represents averages of duplicate determination. hLM t_{1/2} is the half
life in human liver microsomes.
^{b}CL_{int, app} is apparent intrinsic clearance.
Compound 2 corresponds to the following compound:

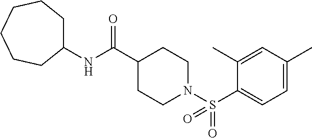 [SMILES: Cc1ccc(S(=O)(=O)N2CCC(C(=O)NC3CCCCCC3)CC2)c(C)c1]

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions, GenBank Accession Numbers, and protocols are cited throughout this application, the disclosures of which are incorporated herein by reference in their entireties for all purpose.

What is claimed is:

1. A compound of Formula I: 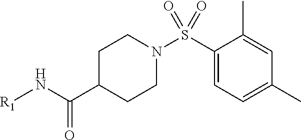
wherein R₁ is selected from the group consisting of substituted or unsubstituted benzothiazol, substituted or unsubstituted pyridyl, substituted or unsubstituted naphthyl, substituted or unsubstituted isoquinolyl, substituted or unsubstituted quinolyl, substituted or unsubstituted phenyl, substituted or unsubstituted alkyl, substituted or unsubstituted cycloalkyl, substituted or unsubstituted aryl, substituted or unsubstituted cycloalkalkyl, substituted or unsubstituted arylalkyl, substituted or unsubstituted heteroarylalkyl, substituted or unsubstituted heteroaryl, and substituted or unsubstituted heterocyclic;
and pharmaceutically acceptable salts and prodrugs thereof.

2. The compound of claim 1, wherein R₁ is selected from the group consisting of substituted cycloalkyl, unsubstituted cycloalkyl, substituted alkyl, unsubstituted naphthyl, and substituted aryl.

3. The compound of claim 1, wherein R₁ is selected from the group consisting of

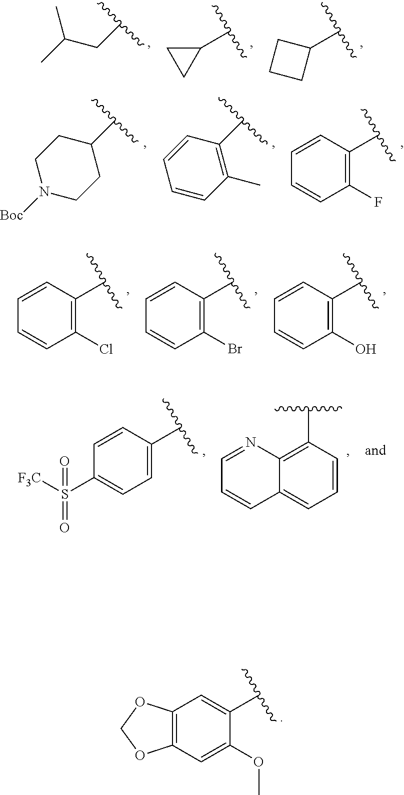

4. The compound of claim 1, wherein the compound is selected from the group consisting of

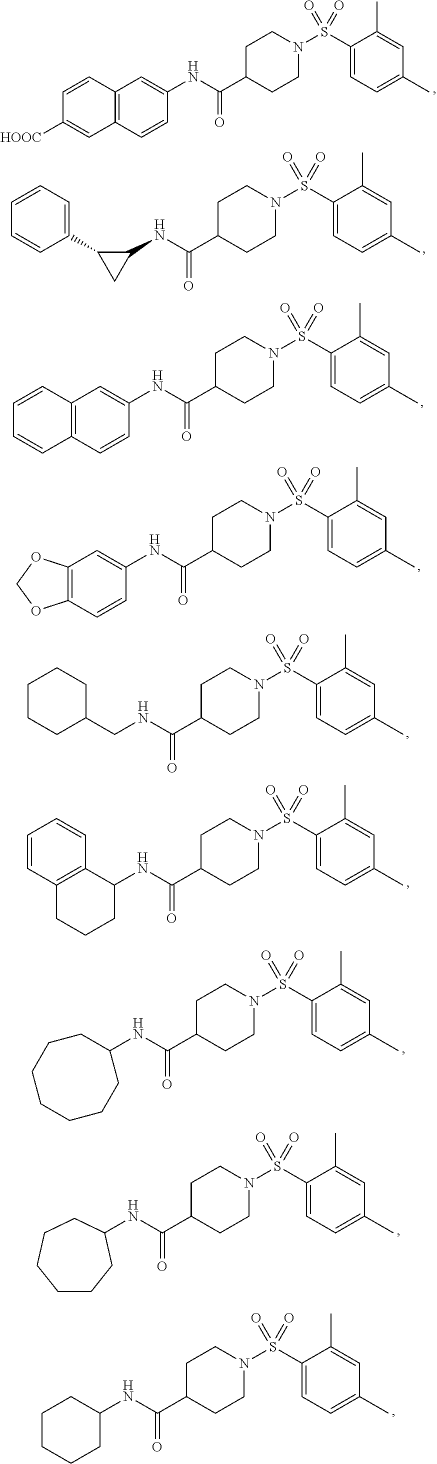

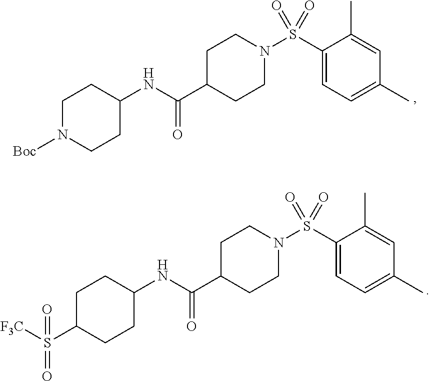 [SMILES: Cc1ccc(S(=O)(=O)N2CCC(C(=O)NC3CCN(C(=O)OC(C)(C)C)CC3)CC2)c(C)c1 ; Cc1ccc(S(=O)(=O)N2CCC(C(=O)NC3CCC(S(=O)(=O)C(F)(F)F)CC3)CC2)c(C)c1]

5. The compound of claim 1, wherein the compound is

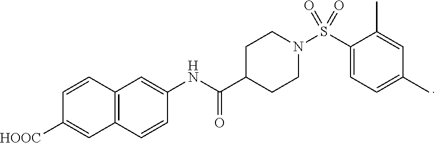 [SMILES: Cc1ccc(S(=O)(=O)N2CCC(C(=O)Nc3ccc4cc(C(=O)O)ccc4c3)CC2)c(C)c1 ; "."]

6. A method for inhibiting the activity of a soluble epoxide hydrolase which comprises contacting the soluble epoxide hydrolase with a compound of Formula I in an amount effective to inhibit soluble epoxide hydrolase activity, wherein Formula I is: 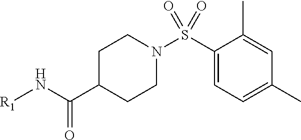
wherein R₁ is selected from the group consisting of substituted or unsubstituted benzothiazol, substituted or unsubstituted pyridyl, substituted or unsubstituted naphthyl, substituted or unsubstituted isoquinolyl, substituted or unsubstituted quinolyl, substituted or unsubstituted phenyl, substituted or unsubstituted alkyl, substituted or unsubstituted cycloalkyl, substituted or unsubstituted aryl, substituted or unsubstituted cycloalkalkyl, substituted or unsubstituted arylalkyl, substituted or unsubstituted heteroarylalkyl, substituted or unsubstituted heteroaryl, and substituted or unsubstituted heterocyclic;
and pharmaceutically acceptable salts and prodrugs thereof.

7. The method of claim 6, wherein R₁ is selected from the group consisting of

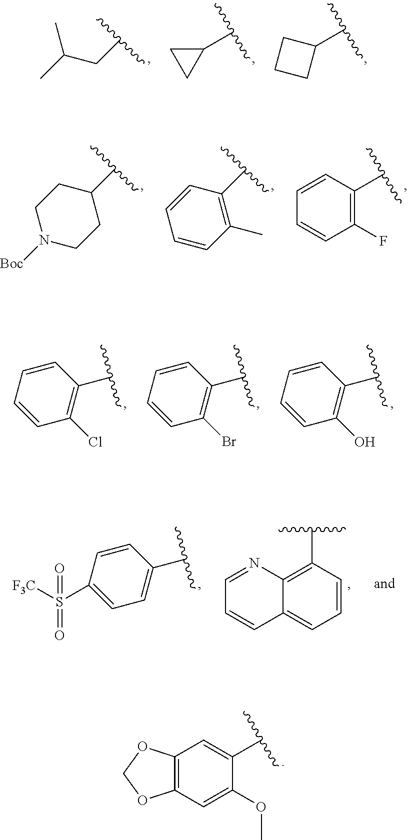

8. The method of claim 6, wherein the inhibition of soluble epoxide hydrolase reduces the metabolism of an epoxyeicosatrienoic acid.

9. The method of claim 6, wherein the soluble epoxide hydrolase is expressed by a cell.

10. The method of claim 9, wherein the cell is a mammalian cell.

11. The method of claim 6, wherein the soluble epoxide hydrolase and compound of Formula I are contacted in vitro.

12. The method of claim 6, wherein the compound of Formula I is selected from the group consisting of:

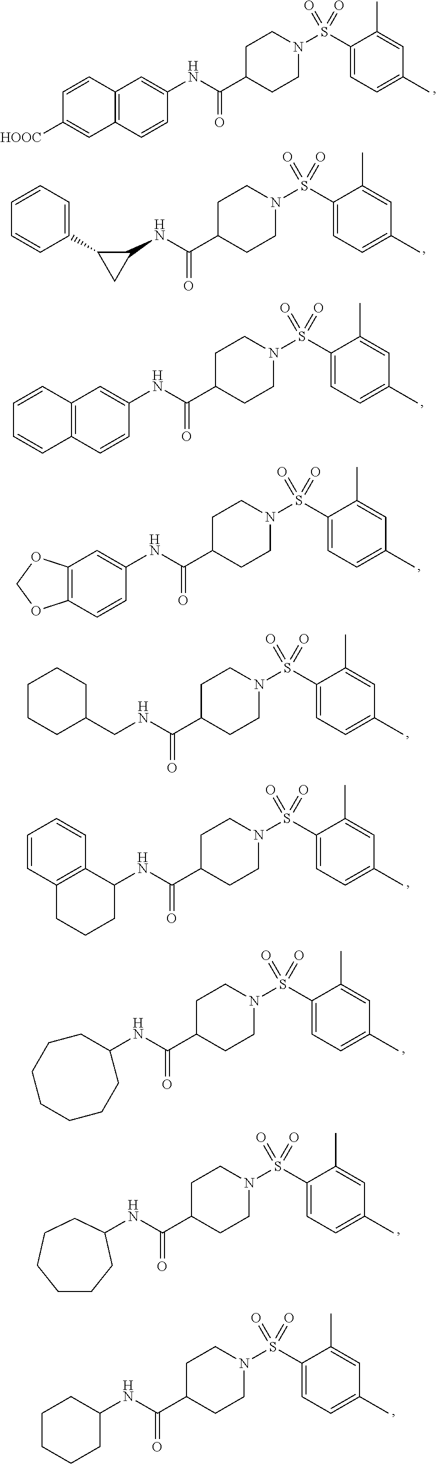

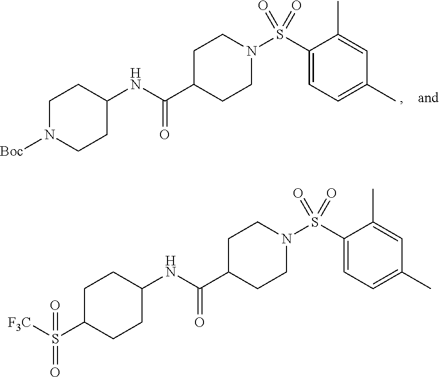 [SMILES: Cc1ccc(S(=O)(=O)N2CCC(C(=O)NC3CCN(C(=O)OC(C)(C)C)CC3)CC2)c(C)c1 ; Cc1ccc(S(=O)(=O)N2CCC(C(=O)NC3CCC(S(=O)(=O)C(F)(F)F)CC3)CC2)c(C)c1]

13. The method of claim 6, wherein the compound of Formula I is

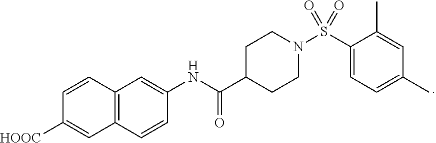 [SMILES: Cc1ccc(S(=O)(=O)N2CCC(C(=O)Nc3ccc4cc(C(=O)O)ccc4c3)CC2)c(C)c1 ; "."]

14. A method for treating a disease related to dysfunction of vasodilation, inflammation, and/or endothelial cell dysfunction in an individual, which method comprises administering to the individual an effective amount of a compound according to Formula I: 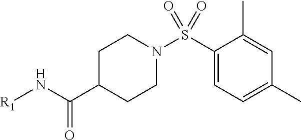
wherein R₁ is selected from the group consisting of substituted or unsubstituted benzothiazol, substituted or unsubstituted pyridyl, substituted or unsubstituted naphthyl, substituted or unsubstituted isoquinolyl, substituted or unsubstituted quinolyl, substituted or unsubstituted phenyl, substituted or unsubstituted alkyl, substituted or unsubstituted cycloalkyl, substituted or unsubstituted aryl, substituted or unsubstituted cycloalkalkyl, substituted or unsubstituted arylalkyl, substituted or unsubstituted heteroarylalkyl, substituted or unsubstituted heteroaryl, and substituted or unsubstituted heterocyclic;
and pharmaceutically acceptable salts and prodrugs thereof.

15. The method of claim 14, wherein R₁ is selected from the group consisting of

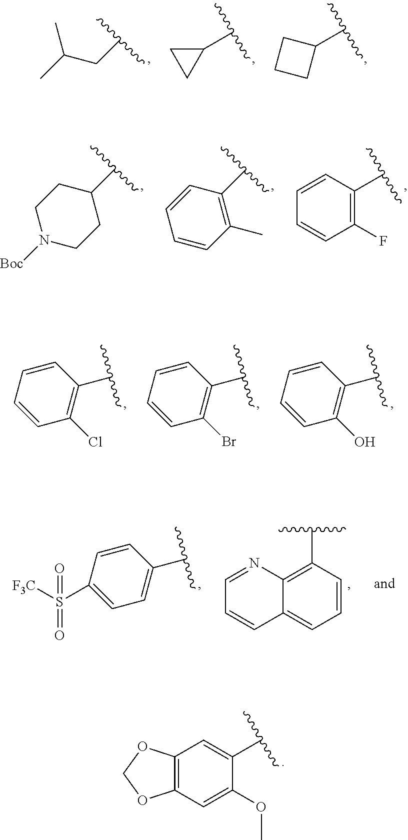

16. The method of claim 14, wherein the disease is hypertension.

17. The method of claim 14, wherein the compound of Formula I is selected from the group consisting of:

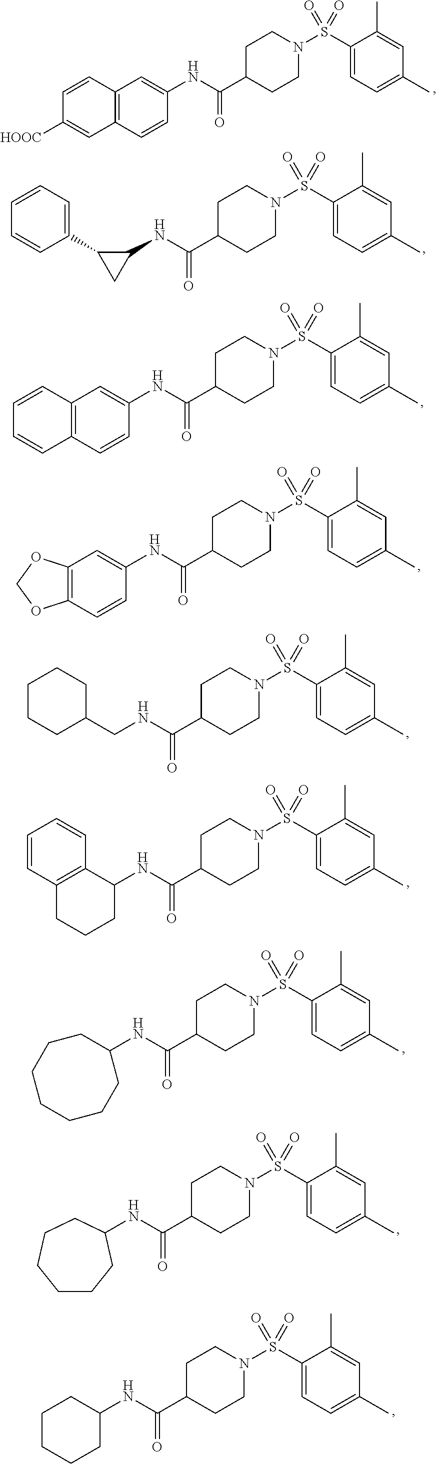

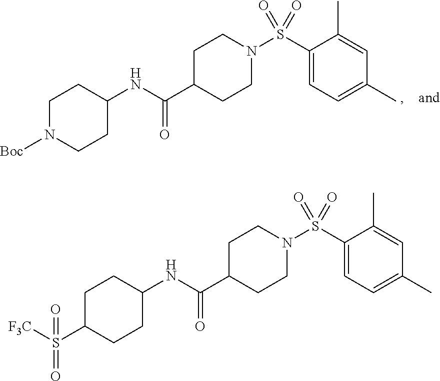 [SMILES: Cc1ccc(S(=O)(=O)N2CCC(C(=O)NC3CCN(C(=O)OC(C)(C)C)CC3)CC2)c(C)c1 ; Cc1ccc(S(=O)(=O)N2CCC(C(=O)NC3CCC(S(=O)(=O)C(F)(F)F)CC3)CC2)c(C)c1]

18. The method of claim 14, wherein the compound of Formula I is

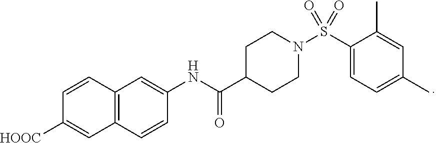 [SMILES: Cc1ccc(S(=O)(=O)N2CCC(C(=O)Nc3ccc4cc(C(=O)O)ccc4c3)CC2)c(C)c1 ; "."]

19. The method of claim 14, wherein the compound is administered to the individual at a dosage effective to achieve a serum concentration of between 0.01 nM and 2 μM.

20. The method of claim 14, wherein the compound is administered to the individual in an amount effective to inhibit the in vitro activity of sEH by at least 5-10%.

21. The method of claim 14, wherein the compound administered to the individual has an IC₅₀ of between 200 nM and 0.01 nM.

22. A pharmaceutical formulation comprising a compound of Formula I: 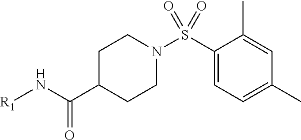
wherein R₁ is selected from the group consisting of substituted or unsubstituted benzothiazol, substituted or unsubstituted pyridyl, substituted or unsubstituted naphthyl, substituted or unsubstituted isoquinolyl, substituted or unsubstituted quinolyl, substituted or unsubstituted phenyl, substituted or unsubstituted alkyl, substituted or unsubstituted cycloalkyl, substituted or unsubstituted aryl, substituted or unsubstituted cycloalkalkyl, substituted or unsubstituted arylalkyl, substituted or unsubstituted heteroarylalkyl, substituted or unsubstituted heteroaryl, and substituted or unsubstituted heterocyclic;
and pharmaceutically acceptable salts and prodrugs thereof.

23. The pharmaceutical formulation of claim 22, wherein R₁ is selected from the group consisting of

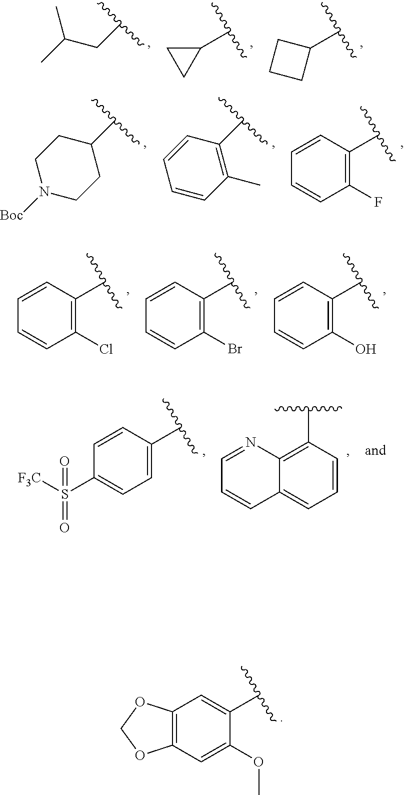

24. The pharmaceutical formulation of claim 22, wherein the compound of Formula I is selected from the group consisting of:

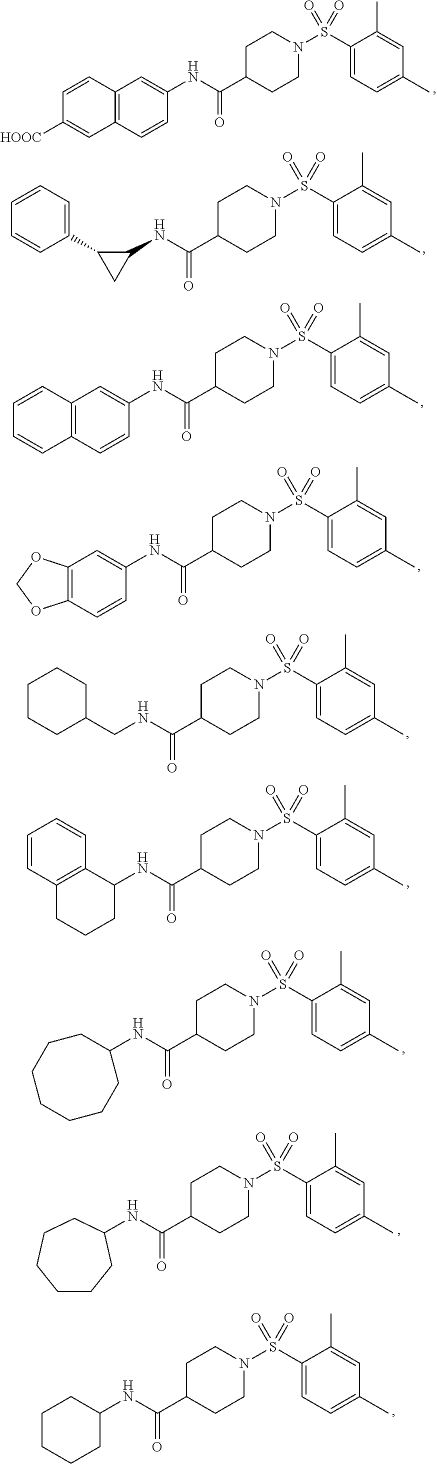

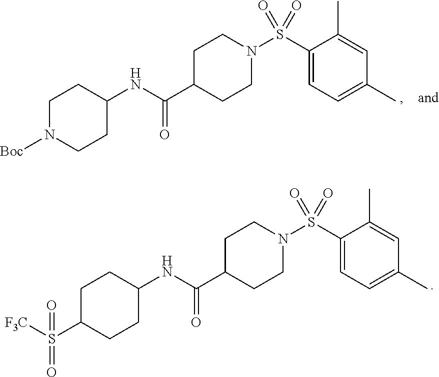 [SMILES: Cc1ccc(S(=O)(=O)N2CCC(C(=O)NC3CCN(C(=O)OC(C)(C)C)CC3)CC2)c(C)c1 ; Cc1ccc(S(=O)(=O)N2CCC(C(=O)NC3CCC(S(=O)(=O)C(F)(F)F)CC3)CC2)c(C)c1]

25. The pharmaceutical formulation of claim 22, wherein the compound of Formula I is 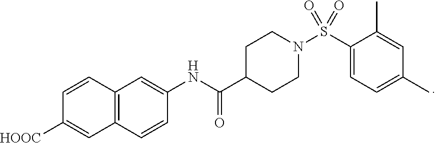 [SMILES: Cc1ccc(S(=O)(=O)N2CCC(C(=O)Nc3ccc4cc(C(=O)O)ccc4c3)CC2)c(C)c1 ; "."]
